(19) United States
(12) Patent Application Publication
(10) Pub. No.: US 2011/0054447 A1
(43) Pub. Date: Mar. 3, 2011

(54) LOCKABLE DRAINAGE CATHETER

(75) Inventors: Thomas R. Johnson, Milford, NH (US); Andrew L. Cote, SR., Merrimack, NH (US); Yem Chin, Burlington, MA (US); Paul M. Scopton, Winchester, MA (US)

(73) Assignee: THERAGENICS CORPORATION, Buford, GA (US)

(21) Appl. No.: 12/548,974

(22) Filed: Aug. 27, 2009

(51) Int. Cl. A61M 25/00

(52) U.S. Cl. 604/540

(57) ABSTRACT

A drainage catheter includes a shaft having a flexible distal portion to be anchored in a cavity of a patient and a proximal portion attached to a hub. The hub includes a side port with a deformable sealing insert having self-sealing passageways for tensioning members, such as nylon filaments, passing through the catheter shaft and exiting the side port. The tensioning members are pulled proximally to deform and thereby anchor the distal portion of the catheter. Excess filament length is wrapped around a resilient pinch washer and held inside a groove in the pinch washer, with the groove thereafter compressed by a C-clip to secure the excess filament length inside the groove.

### BACKGROUND OF THE INVENTION

Flexible catheters are widely used for percutaneous drainage of fluid collections and percutaneous nephrostomy. They are also used for drainage of abscesses, cysts, pleural effusions, empyemas, and other mediastinal collections. In such applications, the catheters are typically inserted either over a previously placed guidewire (so-called Sledinger procedure) or by direct puncture using a trocar cannula. The trocar cannula is removed once the catheter is in place in the patient's body.

Once in position in a body cavity, it is desirable to anchor the catheter before drainage begins. This may be done by forming a restraining portion at the distal end of the catheter in the form of a pigtail, a J-curve or a malecot rib. To reliably anchor and later easily remove the catheter, the restraining portion in the distal end of the catheter should be lockable and unlockable from the proximal end of the catheter, where the catheter protrudes from the body.

A restraining member for locking and unlocking the restraining portion is typically formed by a flexible tensioning member, such as a filament or suture thread, that runs through the length of the catheter and exits a short length from the distal tip through a side hole. The filament then reenters the catheter a short distance proximal from the side hole through a second, similar side hole and runs back to the hub to a point where the tension member can exit. The exit point can be a port or a side arm, from which the tensioning member can be manipulated for the purposes of securing the restraining portion at the distal end. Excess suture material can either be cut and discarded or wrapped around, for example, the side arm, which allows loosening of the lock to reposition the catheter and then securely lock it again.

Several mechanisms have been proposed for locking the filament. For example, the filament read may be locked in place by a compressible bushing compressed by a screw cap. In another approach, the tensioning member may be locked with a pivoting lever that compresses a seal or sleeve.

Moreover, the passageway, through which the tensioning member exits the catheter, should be leak-proof at pressures up to 65 psi or a negative pressure of 2 bars. Various types of sealing elements for sealing that tensioning member has been proposed, such as a compression seal, a molded seal, a self-sealing plug or a sleeve made of, for example, latex.

However, there is still a need to provide a drainage catheter of a simple construction with a liquid-tight seal for the tensioning member that does not require additional sleeves, assemblies or other components, and which retains the tensioning member in its tensioned position after the restraining portion at the distal end of the catheter is in its curled and anchored position, without having to resort to a complex locking mechanism.

### BRIEF SUMMARY OF THE INVENTION

According to one aspect of the invention, a drainage catheter includes a shaft having a flexible distal portion to be anchored in a cavity of a patient and a proximal portion, and a lumen extending through the shaft. The drainage catheter further includes a hub attached to the proximal portion of the shaft, wherein the hub has a port in communication with the lumen, a deformable sealing insert disposed in the port, and a filament running from the distal portion of the shaft at least partially through the lumen of the shaft and having a free end passing through a passageway in the deformable sealing insert and exiting from the hub. The drainage catheter also includes a resilient pinch washer having a circumferential groove configured to receive a wrapped length of the filament that exits from the hub, and a securing member retaining an end section of the wrapped length of the filament on the hub.

According to another aspect of the invention, a method for anchoring a distal portion of a drainage catheter in a cavity of a patient includes the steps of guiding a filament that extends at least partially through a lumen of the catheter through a passageway formed in a deformable sealing insert inserted in a part disposed at a proximal hub section of the catheter, proximally pulling an end section of the filament exiting from the passageway, thereby causing a distal portion of the catheter to deform and anchor in a cavity of a patient, and wrapping the end section inside a circumferential groove of a resilient pinch washer. Thereafter, a securing member is applied which compresses the width of the circumferential groove so as to frictionally secure the end section inside the circumferential groove.

Embodiments of the invention may include one or more of the following features. The wrapped length of the filament may be frictionally secured inside the circumferential groove, such as a V-shaped groove, by providing on a part of the hub, such as a side arm of the hub, an acceptance region having a width that is less than a combined width of the resilient pinch washer and the securing member. Preferably, two filaments exit the passageways proximally. The resilient pinch washer and the securing member are then located side-by-side in the acceptance region and press the securing member against the resilient pinch washer to narrow the width of the circumferential groove. The acceptance region may include a gripping surface constructed to engage with a complementary gripping surface disposed on the securing member, thereby preventing rotation of the securing member relative to the hub. The gripping surface of the acceptance region may have teeth and the complementary gripping surface on the securing member may include mating teeth, thereby locking the securing member on the hub.

The securing member may include at least one through-hole through which the end section(s) of the wrapped length of filament is/are threaded and thereby secured on the securing member. Two end sections of the filament may exit the sealing insert through separate passageways, with the circumferential groove in the pinch washer receiving the two end sections and the securing member receiving and securing both end sections.

In one advantageous embodiment, the sealing insert may barrel-shaped, with a greatest barrel diameter located at the center intermediate the end faces and being greater by at least 10% than an inside diameter of the port, thereby radially compressing the deformable sealing insert and sealing the filament against the passageway following insertion of the sealing insert in the port. The passageways may be formed by piercing the deformable sealing insert with a needle before the deformable sealing insert is inserted in a port located in the proximal hub section, for example, in a side leg of the hub.

The shaft may include markers which are externally applied to or integrally formed with the shaft and are configured to indicate an insertion depth of the catheter in the cavity of the patient. Moreover, the filament may include markers externally applied to or integrally formed with the filament, wherein markers indicate a degree of curling of the flexible catheter tip in the cavity of the patient.

These and other features and advantages of the present invention will become more readily appreciated from the detailed description of the invention that follows.

### BRIEF DESCRIPTION OF THE DRAWINGS

The following figures depict certain illustrative embodiments of the invention in which like reference numerals refer to like elements. These depicted embodiments are to be understood as illustrative of the invention and not as limiting in any way.

FIG. 1 is a perspective view of the drainage catheter according to the present invention, with a portion of the tensioning member extending outside the distal end to lock the distal end in a curled configuration;

FIG. 2 is a side elevational view in cross-section of the drainage catheter shown in FIG. 1, with the distal end in the curled configuration; and

FIG. 3 is an exploded perspective view of the proximal end of the drainage catheter with tensioning member, pinch washer and sealing plug.

### DETAILED DESCRIPTION OF THE INVENTION

Throughout all the figures, same or corresponding elements may generally be indicated by same reference numerals. These depicted embodiments are to be understood as illustrative of the invention and not as limiting in any way. Rather, numerous alterations and modifications are possible in the context of the present disclosure, in particular those variants, elements and combinations which, for example through combination or modification of individual features or elements or method steps in conjunction with the features or elements or method steps which are described in the general or specific part of the description and are contained in the claims and/or the drawing. It should also be understood that the figures are not necessarily to scale and that the embodiments are sometimes illustrated by graphic symbols, phantom lines, diagrammatic representations and fragmentary views. In certain instances, details which are not necessary for an understanding of the present invention or which render other details difficult to perceive may have been omitted.

The present invention is directed to a drainage catheter of simple construction which provides a simple, yet effective seal between a monofilament tensioning member and a sealing plug while at the same time proximally retaining and locking the tensioning member to prevent the distal tip of the drainage catheter from being unintentionally removed from a patient.

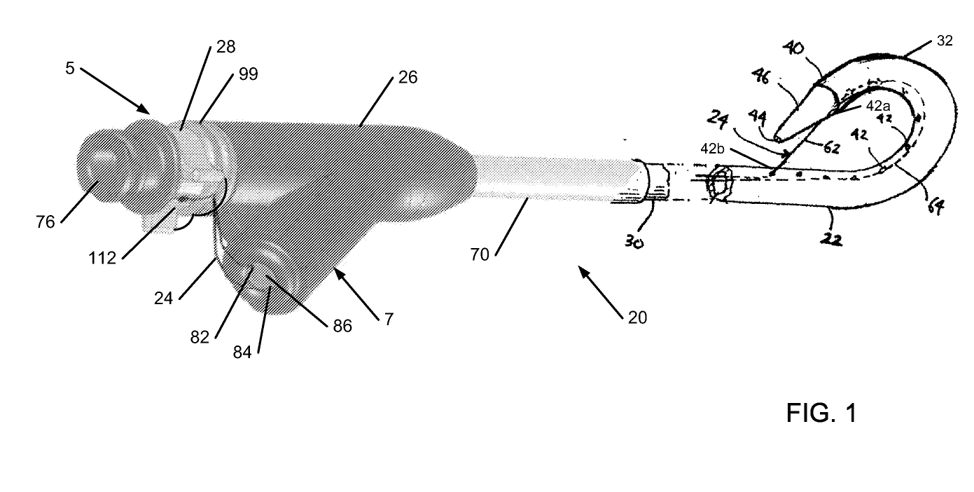

Turning now to the drawing, and in particular to FIG. 1, there is shown in a schematically simplified manner a drainage catheter 20 having an elongated hollow drainage tube 22, a proximal fitting or hub 26, a tensioning member 24, and a securing member 28. At the outset, drainage catheter 20 will be shown and described as a locking-type catheter wherein the flexible tensioning member 24 affirmatively alters the shape of the distal end 32 of the drainage tube 22 to retain the drainage tube 22 inside the body cavity of a patient. However, it is likewise contemplated that the distal end of the drainage catheter may be at least partially pre-formed or pre-altered to facilitate shaping the tip to a looped, curled, coiled or other desired configuration. It will be understood that, if the distal end 32 of the drainage tube 22 is pre-shaped, a stiffening cannula can be used to insert the drainage catheter into a patient's body.

The elongated, preferably flexible hollow drainage tube 22 includes a proximal end 30 and a distal end 32 and is preferably formed from a plastic and/or polymer, preferably polyurethane. The elongated hollow drainage tube must be non-reactive to bodily fluids coming into contact with the drainage tube, while at the same time remaining malleable and flexible.

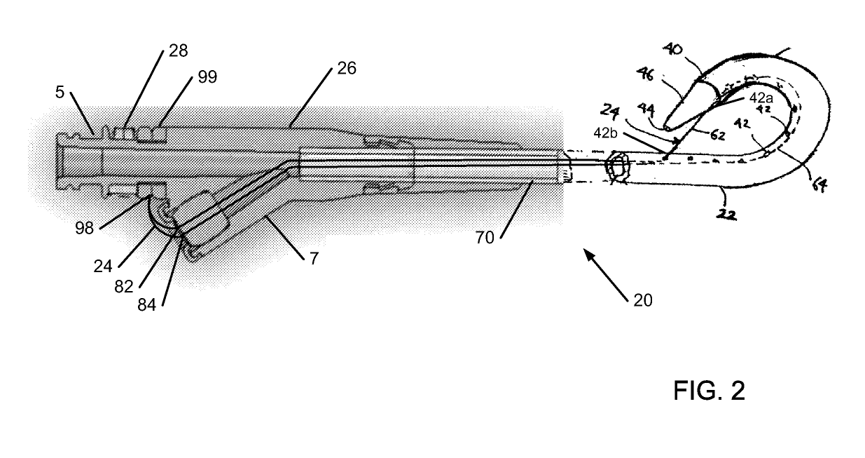

The distal end 32 includes a flexible tip 40 with drainage openings 42 and a fluid opening 44 at the distal end of conical portion 46. The fluid opening 44 extends through a conical portion 46 of the flexible tip 40 and is in fluid communication with the inside of the elongated hollow drainage tube 22. As mentioned above, the flexible tip 40 is capable of assuming a number of configurations, such as a J-curl, a pigtail loop (FIGS. 1 and 2), or other configurations. The degree of curl depends on the construction and orientation of drainage catheter 20, including the specific threading pattern of tensioning member 24, the amount of tension placed on tensioning member 24, the length of elongated hollow drainage tube 22, the flexibility of the catheter material, etc. It will also be understood that the flexible tip 40 may take any shape that facilitates insertion of the drainage catheter into the patient's body.

Fluid drained from the patient's body cavity flows into elongated hollow drainage tube 22 through fluid opening 44 and additional drainage openings 42 upon insertion of drainage catheter 20 into a patient's body cavity. The number and location of the drainage openings 42, which may be small holes drilled into or otherwise applied to one side of elongated hollow drainage tube 22 may be selected to accommodate a specific drainage application. One of the drainage openings 42 may also operate as a threading hole for tensioning member 24, as shown in FIGS. 1 and 2 and to be described below.

Tensioning member 24 shown in FIGS. 1 and 2 includes a first segment 62 extending outside the drainage tube 22 and a second segment 64 extending inside the drainage tube 22. The first segment 62 of tensioning member 24 exits the drainage tube 22 through a distal drainage opening 42a and reenters the drainage tube 22 through a proximal drainage opening 42b. When the tensioning member is pulled proximally, the first segment becomes shorter, thereby pulling the distal drainage opening 42a towards the proximal drainage opening 42b and causing the distal end 32 to curl. Tensioning member 24 is preferably constructed from a nylon monofilament; however, those of ordinary skill in the relevant art will appreciate that tensioning member 24 may also be multi-stranded and may furthermore be made from wire, stainless steel, plastic or other polymers, which do not significantly stretch when tensioned.

As shown in a perspective view in FIG. 1 and in a side elevational view in FIG. 2, a substantially Y-shaped hub 26 may include an annular sleeve 70 or a molded strain relief located near the proximal end 30 of the hollow drainage tube 22, a drainage leg 5 with a drainage passageway 78, and a side leg 7 with a passageway for the tensioning members 24. The drainage leg 5 may have a threaded connecting port 76 with a compression fitting (not shown).

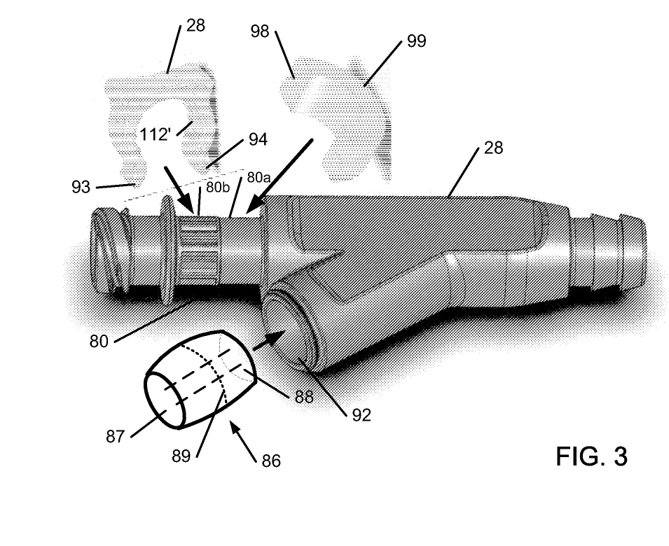

As indicated in FIG. 1 and more clearly shown in the cross-sectional view in FIG. 2 and the exploded view in FIG. 3, side leg 7 has an opening adapted and sized to accept a sealing insert 86 with dedicated sealing passageways 82, 84 for the tensioning members 24. As shown in FIG. 3, the sealing insert 86 is barrel-shaped, with two end faces 87, 88 and a midsection 89 whose diameter is greater than the diameter of the end faces 87, 88. The dimensions of the sealing insert 86 are selected to create uniform compression with respect to the tensioning members 24 along substantially the entire length of the passageways 82, 84 after the sealing insert 86 is inserted into bore 92 in side leg 7. In the particular illustrated exemplary embodiment, the sealing insert 86 has been designed to concentrate the compression forces in such a way as to not only maximize sealing, but to also minimize the pulling forces required to pull the tensioning members 24 when shaping the pigtail in the distal end 32.

In one exemplary embodiment, assuming a 0.250″ diameter bore 92 in the side leg 7, FEA (Finite Element Analysis) yielded optimal uniform compression with a sealing insert 86 having a length of 0.25″, an outer diameter of 0.200″ at the two end faces 87, 88 and a greatest diameter of 0.260″ at a location 89 intermediate the two end faces 87, 88. The uniform compression attained throughout the passageways 82, 84 provides excellent sealing between the sealing insert and the tensioning members 24 at the 65 psi test limit while reducing the pulling force required to activate the pigtail loop to remain under 0.25 lbs.

The side arm in the fitting that accepts the sealing insert 86 is configured with a slight relief angle of ½ to 1 degree per side tapering from the base of the bore at 0.250″ out. The sealing insert 86 is retained feature in bore 92 by a cap which is snapped in place in a mating groove on side leg 7. The cap has a through bore of 0.156″ and a large flat area in contact with the sealing insert 86, without adding any compressive force. The large flat contact area of the cap prevents a rocking or a gimbal effect as result of the barrel shape of the sealing insert.

A silicone material, such as LIM silicone 6030, was selected for resistance to setting under compression, which could adversely affect sealing and uniformity of the pull force. The material was selected on the basis of achieving a balance between the sealing forces and the pull forces, while reliably sealing at pressures reaching 65 psi and having a pull force not exceeding 0.25 lbs. The shape of the plug and the material has been designed to work with a Nylon 3/0 monofilament suture (0.009″ dia.). However, other materials with similar physical properties could also be used.

The sealing insert 86 is manufactured without molded or predrilled holes, with the filament being threaded through the seal by attaching the filament to a needle and pushing the needle through the seal, dragging the filament with it. It has been found that piercing the silicone material a needle of suitable diameter in relation to the diameter of the filament creates a self-sealing passage for the filament. The diameter of the needles used with the aforedescribed Nylon 3/0 monofilament suture (0.009″ dia.) may range from 0.020″ to 0.040″. The filament is preferably a monofilament which has been found to exhibit superior sealing properties. The filament may be attached to a needle by threading the filament through the eye of the needle or by fixedly attaching the needle to the filament, for example, by crimping. There is no additional sealing member or sleeve necessary for the outer or inner portion of hub proximate the entrance or exit points of filament 24.

The hub 26 has a filament accepting region 80 which, as shown in FIGS. 1 and 3, may be disposed on the proximal end of drainage leg 5. However, the filament accepting region 80 may also be placed on the side leg 7, if suitably dimensioned. In the illustrated exemplary embodiment shown more clearly in FIG. 3, the filament accepting region 80 has a first region 80a for a pinch washer 99 and a second section 80b adapted to receive a securing member 28, such as the depicted C-clip. The second region 80b may include a gripping surface 112 adapted to engage with a complementary gripping surface 112′ disposed on an inner surface of the securing member 28. The gripping surfaces 112, 112′ may be formed so as to operate as a ratchet, which may lock in one or in both rotational directions of the securing member 28 relative to the hub 26. However, gripping surfaces 112, 112′ may likewise include other ratcheting teeth configurations, roughened finish, dimpled surfaces or any other suitable gripping surfaces. The securing member 28 and the pinch washer 99 cooperate with filaments 24, as described in more detail below.

As shown in FIGS. 1 to 3, pinch washer 99 has a groove 98 that receives excess filament length when the filament is pulled to curl distal end 32 of drainage tube 22. The groove 98 frictionally engages filament to resist tension exerted on the filament by the curled distal end 32. The combined widths of the securing member 28 and the pinch washer 99 are selected to be slightly greater than the overall width of the accepting region 80, i.e. the combined width of first region 80a and second region 80b, so that the pinch washer 99 is compressed when the securing member 28 is secured in the second region 80b. A preferred material for the pinch washer 99 is Silicone with 60A durometer, which provides enough friction to hold the filament firmly in the groove 98, in particular with the additional compressive force applied on the pinch washer 99 by the clipped-on “C”-clip. It will be understood that other elastomeric materials aside from silicone may also be used. The leading edges of the groove are radiuses to help guide the filament during the coiling process; the gap is about 0.004″ wide and has a depth of about 0.065″.

The securing member 28, or C-clip, in the illustrated exemplary embodiment has two through-holes 93, 94 through which end sections of filament 24 are threaded and secured by a knot. The securing member 28 also has a radiused leading edge to guide the clip onto the hub, to release the locking action and to facilitate removal. Securing member 28 simply snaps onto hub 26. The securing member 28 may be rotated until the tensioning members 24 are taught, thereby locking the flexible tip 40 in the desired, typically curled, configuration.

In another embodiment, the securing member or C-clip may only have a single through-hole, i.e. either through-hole 93 or 94, to improve control when the tensioning members 24 are wrapped around pinch washer 99.

In operation, drainage catheter 20 is inserted into a patient's body cavity. This step may be performed with the aid of a stiffening cannula, which is inserted through the drainage leg 5 of hub 26 into the elongated hollow drainage tube 22. Once inside a body cavity, fluid enters the elongated hollow drainage tube 22 through the fluid opening 44 disposed in the flexible tip 40, as well as through drainage openings 42. However, since the proximal ends of filament 24 are sealingly and slidably retained in the filament passageways 82 and 84 in side leg 7 of hub 26, fluid does not leak through the side leg 7, but instead exits the drainage leg 5 after opening a drainage passageway in drainage leg 5.

Upon proper catheter positioning, the portion of filament 24 exiting the filament passageways 82 and 84 is then retracted, thus tensioning first segment 62 and second segment 64 of filament 24 and altering the orientation of flexible tip 40 to the desired configuration. Once the tip configuration is properly altered to retain the drainage catheter 20 inside a patient's body cavity, the excess length of filament 24 is wrapped around pinch washer 99 inside groove 98. Securing member 28 with the attached end of the remainder of filament 24 is then snapped onto section 80b of filament accepting region 80 to lock the filament 24 in its tensioned and tip-altering configuration.

Once drainage is complete, the clip can be removed and the sutures un-wound to release the locking mechanism to reposition the catheter and then re-lock the catheter in place. Alternatively, when the drainage catheter is to be completely removed from the patient's body, one or both ends of filament 24 extending outside the patient's body are severed, thus restoring the flexible tip to its uncurled state and permitting filament 24 to be removed from drainage catheter 20. The drainage catheter 20 may be removed from a patient's body with or without using a stiffening cannula to facilitate retraction and removal of drainage catheter 20.

The body of the catheter may be provided with axially spaced markings and/or numerical indicia spaced, for example, at 1 and 5 cm intervals to indicate to the physician placing the catheter the location of the distal tip within the patient. In addition, a tether limiting the pull length of the filament may be included, to ensure that the pigtail is fully formed, while preventing buckling or accordioning of the distal end 32. Accordioning may also be prevented by making the catheter shaft of two or more materials having different durometer values, with a higher durometer material forming the distal end to facilitate placement of the catheter. It has been observed that catheters of a lower uniform durometer tend to buckle or accordion as pressure is applied to push the catheter into place. The design with two sections of different durometer will achieve the desired effect of having good column strength to ease the placement of the catheter while still retaining a soft compatible main shaft for patient comfort and reduced chance of kinking.

In another embodiment, one or more markers may be applied to or embedded at least in the filament section exiting the passageways 82, 84, with the markers providing an indication when the distal end attains its intended curled, e.g. pigtail, shape when the filament 24 is pulled out of the passageways 82, 84.

While the invention has been illustrated and described in connection with currently preferred embodiments shown and described in detail, it is not intended to be limited to the details shown since various modifications and structural changes may be made without departing in any way from the spirit and scope of the present invention. The embodiments were chosen and described in order to explain the principles of the invention and practical application to thereby enable a person skilled in the art to best utilize the invention and various embodiments with various modifications as are suited to the particular use contemplated.

What is claimed as new and desired to be protected by Letters Patent is set forth in the appended claims and includes equivalents of the elements recited therein:

What is claimed as new and desired to be protected by Letters Patent of the United States is:

1. A drainage catheter comprising:
a shaft having a flexible distal portion to be anchored in a cavity of a patient and a proximal portion, and a lumen extending through the shaft; and
a hub attached to the proximal portion of the shaft, the hub comprising:
a port in communication with the lumen;
a deformable sealing insert disposed in the port;
a filament running from the distal portion of the shaft at least partially through the lumen of the shaft and having a free end passing through a passageway in the deformable sealing insert and exiting from the hub;
a resilient pinch washer having a circumferential groove configured to receive a wrapped length of the filament that exits from the hub; and
a securing member retaining an end section of the wrapped length of the filament on the hub.

2. The catheter of claim 1, wherein the hub comprises an acceptance region having a width that is less than a combined width of the resilient pinch washer and the securing member, said resilient pinch washer and the securing member located side-by-side in the acceptance region so as to press the securing member against the resilient pinch washer and frictionally securing the wrapped length of the filament inside the circumferential groove.

3. The catheter of claim 2, wherein the acceptance region comprises a gripping surface constructed to engage with a complementary gripping surface disposed on the securing member, thereby preventing rotation of the securing member relative to the hub.

4. The catheter of claim 3, wherein the gripping surface of the acceptance region comprises teeth and the complementary gripping surface on the securing member comprises mating teeth.

5. The catheter of claim 1, wherein a width of the circumferential groove of the resilient pinch washer is smaller than a diameter of the filament.

6. The catheter of claim 5, wherein the circumferential groove is V-shaped.

7. The catheter of claim 1, wherein the securing member comprises at least one through-hole for retaining the end section of the wrapped length of filament.

8. The catheter of claim 1, wherein the proximal portion of the shaft and the distal portion of the shaft are made of materials having different durometer values, with the distal portion of the shaft having a greater durometer value than the proximal portion of the shaft.

9. The catheter of claim 1, wherein the sealing insert is barrel-shaped with end faces, with a greatest barrel diameter located intermediate the end faces and being greater by at least 10% than an inside diameter of the port, thereby radially compressing the deformable sealing insert and sealing the filament against the passageway following insertion of the sealing insert in the port.

10. The catheter of claim 1, wherein the shaft comprises markers applied to or integrally formed with the shaft, said markers indicating an insertion depth of the catheter in the cavity of the patient.

11. The catheter of claim 1, wherein the filament comprises markers applied to or integrally formed with the filament, said markers indicating a degree of curling of the flexible catheter tip in the cavity of the patient.

12. The catheter of claim 1, wherein the port is disposed in a side leg of the hub.

13. The catheter of claim 1, wherein two end sections of the filament exit the sealing insert through separate passageways, with the circumferential groove in the pinch washer receiving the two end sections, which the securing member receiving and securing the two end sections.

14. A method for anchoring a distal portion of a drainage catheter in a cavity of a patient, comprising the steps of:
guiding a filament that extends at least partially through a lumen of the catheter through a passageway formed in a deformable sealing insert inserted in a part disposed at a proximal hub section of the catheter,
proximally pulling an end section of the filament exiting from the passageway, thereby causing a distal portion of the catheter to deform and anchor in a cavity of a patient,
wrapping the end section inside a circumferential groove of a resilient pinch washer,
applying a securing member configured to compress a width of the circumferential groove so as to frictionally secure the end section inside the circumferential groove.

15. The method of claim 14, wherein applying the securing member comprises placing the securing member and the resilient pinch washer side-by-side in an acceptance region having a width that is less than a combined width of the resilient pinch washer and the securing member.

16. The method of claim 14, wherein the passageway is formed by piercing the deformable sealing insert with a needle before insertion of the deformable sealing insert in the proximal hub section.
